(19) United States
(12) Patent Application Publication
(10) Pub. No.: US 2026/0117173 A1
(43) Pub. Date: Apr. 30, 2026

(54) METHOD FOR MESENCHYMAL PHENOTYPE REVERSION IN PRIMARY HUMAN CORNEAL ENDOTHELIAL CELLS

(71) Applicants: UNIVERSITÀ DEGLI STUDI DI MODENA E REGGIO EMILIA, Modena (MO) (IT); HOLOSTEM S.R.L., Modena (MO) (IT)

(72) Inventors: Graziella PELLEGRINI, Modena (MO) (IT); Eleonora MAURIZI, Modena (MO) (IT)

(73) Assignees: UNIVERSITÀ DEGLI STUDI DI MODENA E REGGIO EMILIA, Modena (MO) (IT); HOLOSTEM S.R.L., Modena (MO) (IT)

(21) Appl. No.: 19/115,977

(22) PCT Filed: Sep. 26, 2023
(86) PCT No.: PCT/EP2023/076499

§ 371 Date: Mar. 27, 2025

(30) Foreign Application Priority Data
Sep. 29, 2022 (IT) 102022000020061

(51) Int. Cl. C12N 5/079

(52) U.S. Cl. CPC C12N 5/0621; C12N 2501/11; C12N 2501/115; C12N 2501/727; C12N 2506/13

(57) ABSTRACT

The present invention refers to an in vitro or ex vivo method for reversing and/or reducing and/or inhibiting the endothelial to mesenchymal transition (EnMT) process in corneal endothelial cells, preferably in primary and/or human corneal endothelial cells, more preferably primary human corneal endothelial cells (HCEnCs), said method comprising the step of incubating said cells with at least one inhibitor of glycogen synthase kinase 3 (GSK-3).

### FIELD OF THE INVENTION

The present invention refers to regenerative medicine field. In particular it refers to a method for reversing and/or reducing and/or inhibiting the endothelial to mesenchymal transition (EnMT) process in corneal endothelial cells, preferably in primary and/or human corneal endothelial cells, more preferably primary human corneal endothelial cells (HCEnCs), said method comprising the step of incubating said cells with at least one inhibitor of glycogen synthase kinase 3 (GSK-3).

### BACKGROUND ART

Corneal endothelium is the innermost epithelial layer of the cornea, retaining the fundamental role of maintaining its transparency and clear vision by regulation of nutrients and fluid exchange across the whole cornea (Català et al., 2021).

However, differently from the corneal epithelium, corneal endothelium has a poor regenerative capacity, a feature that limits its capacity to heal the damages in vivo but also the opportunity to be regenerated in vitro.

A dysfunction in corneal endothelium represents one of the main indications for corneal transplantation, the most frequent type of graft performed worldwide for which, however, only one person in 70 in need can be transplanted. Given an estimated 12.7 million people awaiting for a corneal transplantation globally (Gain et al., 2016), the development of an alternative strategy to regenerate corneal endothelium would be highly beneficial.

Human corneal endothelium is composed by a sealed pattern of hexagonal human corneal endothelial cells (HCEnCs) that guarantees an optimal exchange of liquids with the neighbouring tissues and, consequently, a clear vision. Any loss of such a hexagonal phenotype following endothelial to mesenchymal transition (EnMT) represents a major hindrance that prevents primary HCEnCs to be cultured beyond the second or third passage (Frausto et al., 2020). This issue does not allow to expand HCEnCs population in vitro in order to characterise its culture, study its molecular mechanisms and consequently develop a regenerative cell therapy approach that would overcome corneal transplantation.

It is still felt the need of a method for inhibiting or reversing endothelial to mesenchymal transition (EnMT) in HCEnCs cultures or populations.

### SUMMARY OF THE INVENTION

The method developed herein allowed for the first time to identify a specific factor able to reverse the EnMT process in primary cultured HCEnCs, which re-acquired their characteristic hexagonal morphology and ZO-1 marker expression.

It is therefore an object of the invention an in vitro or ex vivo method for reversing and/or reducing and/or inhibiting the endothelial to mesenchymal transition (EnMT) process in corneal endothelial cells, preferably in primary and/or human corneal endothelial cells, more preferably primary human corneal endothelial cells (HCEnCs),

said method comprising the step of incubating said cells with at least one inhibitor of glycogen synthase kinase 3 (GSK-3).

In a preferred embodiment, said corneal endothelial cells are primary corneal endothelial cells, more preferably primary human corneal endothelial cells (HCEnCs).

Preferably, the corneal endothelial cells are at any passage.

In an embodiment, said method does not comprise the addition of any molecule able to induce endothelial to mesenchymal transition (EnMT), in particular it does not comprise the addition of any factor able to induce EnMT, such as for example TGF-β.

Another object of the invention is an in vitro or ex vivo method for culturing and/or expanding and/or regenerating corneal endothelial cells comprising the method as defined above.

A further object of the invention is an inhibitor of glycogen synthase kinase 3 (GSK-3) for use in reversing and/or reducing and/or inhibiting the endothelial to mesenchymal transition (EnMT) process and/or for use in pharmaco- or toxicology studies and in the treatment of a disease or condition associated to dysfunctional or damaged corneal endothelial cells, preferably wherein the disease or condition is selected from the group consisting of: Fuch's dystrophy, iridocorneal endothelial syndrome, posterior polymorphic dystrophy, congenital hereditary endothelial corneal dystrophy, corneal dystrophy, and advanced endothelial failure in corneal transplantation and/or in regenerative medicine cell therapy approach.

Another object of the invention is the use of an inhibitor of GSK-3 for reversing and/or reducing and/or inhibiting the EnMT process in corneal endothelial cells, preferably in primary and/or human corneal endothelial cells, more preferably primary human corneal endothelial cells (HCEnCs), preferably said cells being in culture.

Preferably, the corneal endothelial cells are at any cell passage.

In an embodiment, the corneal endothelial cells, preferably primary and/or human corneal endothelial cells, more preferably primary human corneal endothelial cells (HCEnCs), are previously isolated from a human subject, preferably said subject having an age above or equal to 60 years.

Preferably, the inhibitor is selected from the group consisting of: CHIR99021, LiCl, Li2CO3, BIO ((2′Z,3′E)-6-bromoindirubin-3′-oxime), TD114-2, campalone, TWS119, CBM1078, SB216763, 3F8 (TOCRIS), AR-A014418, FRATide, indirubin 3′-oxime, CHIR98014, Kenpaullone, TDZD-8, BIO-acetoxime, Bis-7-indolylmaleimide, SB415286, Ro3303544, CP21R7, LY2090314, and L803 or an analogue thereof, such as 6-bromoindirubin-3′-oxime (BIO).

Preferably the inhibitor of GSK-3 is used in combination with at least one molecule that stimulates corneal endothelial cells proliferation and regeneration, preferably said molecule is EGF or EGF analogue or FGF-2 or FGF-2 analogue, or is used in combination with molecular tools aiming at enhancing cells proliferation, such as those introducing a pro-proliferative gene or exploiting RNA interference mechanisms and CRISPR. Preferably, such molecular tools aiming at enhancing cells proliferation are not tools able to induce EnMT.

Preferably the cells are incubated with an inhibitor of GSK-3 during cell amplification for 2 hours-10 days.

Preferably the cells are primary HCEnCs, preferably at any passage.

Preferably the primary HCEnCs are in culture and the incubation of said primary HCEnCs culture with an inhibitor of GSK-3 consists in the addition of the inhibitor to the culture media or culturing the cells with a culture medium comprising the inhibitor.

The reversing and/or reducing and/or inhibiting the EnMT process preferably comprises reversing cell mesenchymal phenotype to endothelial phenotype and/or restoring the cell phenotype to a cell polygonal or hexagonal morphology and/or reverting cell elongated morphology and/or a variation in the expression of EnMT markers, e.g. ZO-1 and/or α-SMA.

A further object of the invention is a medicament comprising the corneal endothelial cell obtainable by the method according to any one of claim 1-2 or 5-8, preferably for medical or pharmaco- or toxicology use, particularly for use in the treatment of a disease or condition associated to dysfunctional or damaged corneal endothelial cells, preferably wherein the disease or condition is selected from the group consisting of: Fuch's dystrophy, iridocorneal endothelial syndrome, posterior polymorphic dystrophy, congenital hereditary endothelial corneal dystrophy, corneal dystrophy, and advanced endothelial failure in corneal transplantation, for use in transplantation and/or in regenerative medicine cell therapy approach.

### DETAILED DESCRIPTION OF THE INVENTION

GSK-3 is preferably characterized by the sequences discloses in NCBI with the following Accession nos.:
NCBI Gene ref.: NG_012922.1
NCBI RNA ref.: NM_001146156.2
NCBI protein ref.: NP_001139628.1
(see also https://www.ncbi.nlm.nih.gov/gene/2932).

The inhibitor of GSK-3 can be used both in vitro and in vivo. In vitro it makes it possible to amplify the cells of the corneal endothelium while maintaining the correct phenotype. These cultures can be used e.g. for the study of drugs or to expand the availability of cells needed for the treatment of diseases (Kinoshita et al. 2018 New Eng. J. Med).

The inhibitor of GSK-3, such as CHIR, can be used in combination with other molecules that stimulate corneal endothelial cells proliferation and regeneration, before or after their application. In particular, the inhibitor of GSK-3, such as CHIR, can be used in combination with EGF and EGF analogues (Pizzuto et al., 2022b), FGF-2 and FGF-2 analogues (Eveleth et al., 2020; Eveleth and Tremblay, 2022; Pizzuto et al., 2022a; Weant et al., 2021) that were proved to stimulate wound healing of corneal endothelium, even after surgical interventions such as descemetorhexis and Descemet's stripping.

Similarly, the inhibitor of GSK-3, such as CHIR, can be used together with molecular tools aiming at enhancing cells proliferation, such as those introducing a pro-proliferative gene or exploiting RNA interference mechanisms and CRISPR (Arras et al., 2021).

In the present invention, the inhibitor of GSK-3 may be selected from the following:

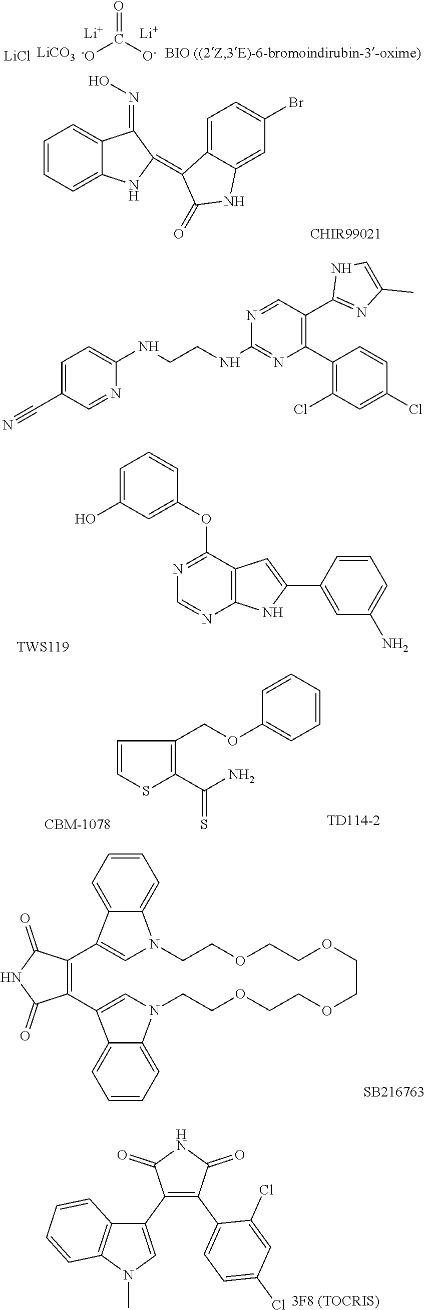

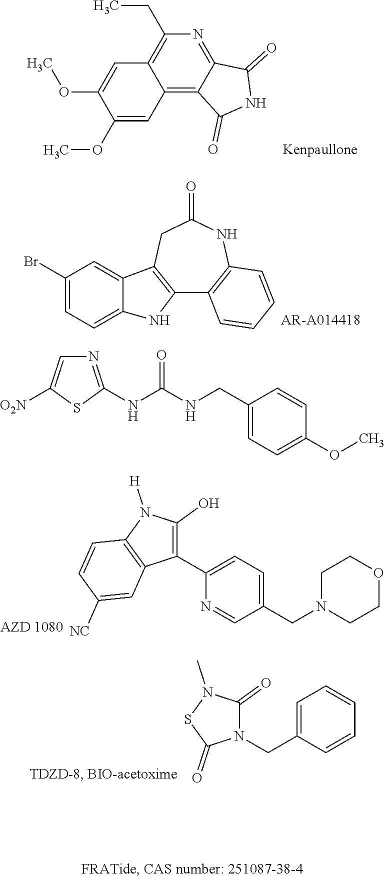

GSK3B-Interacting Protein (IP), NCBI: NP_001258835.1, NP_001258834.1, NP_001258833.1, NP_057566.2
Axin GSK Interaction Domain (GID) 381-405, NCBI Axin: NP_003493.1, NP_851393.1, XP_011520984.1, XP_011520985.1, XP_011520988.1

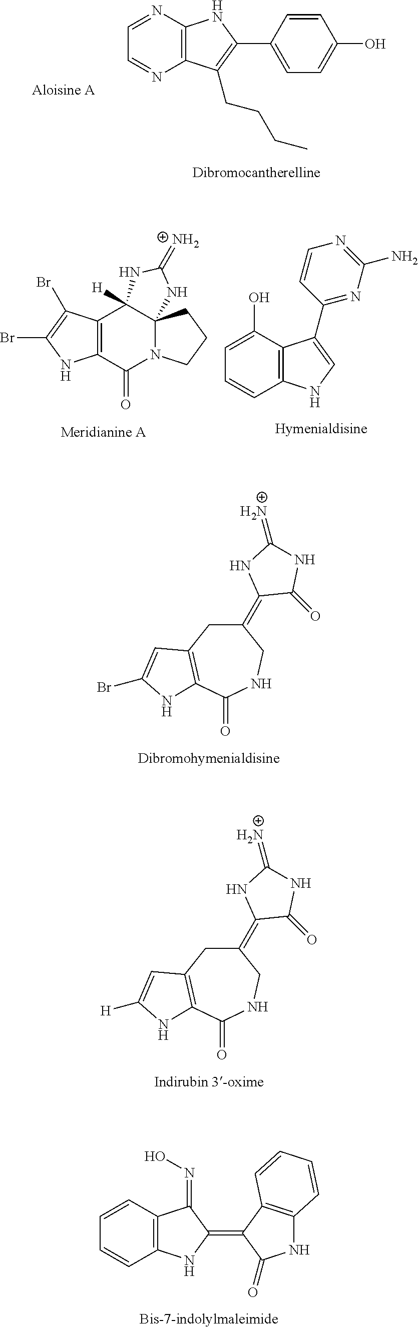

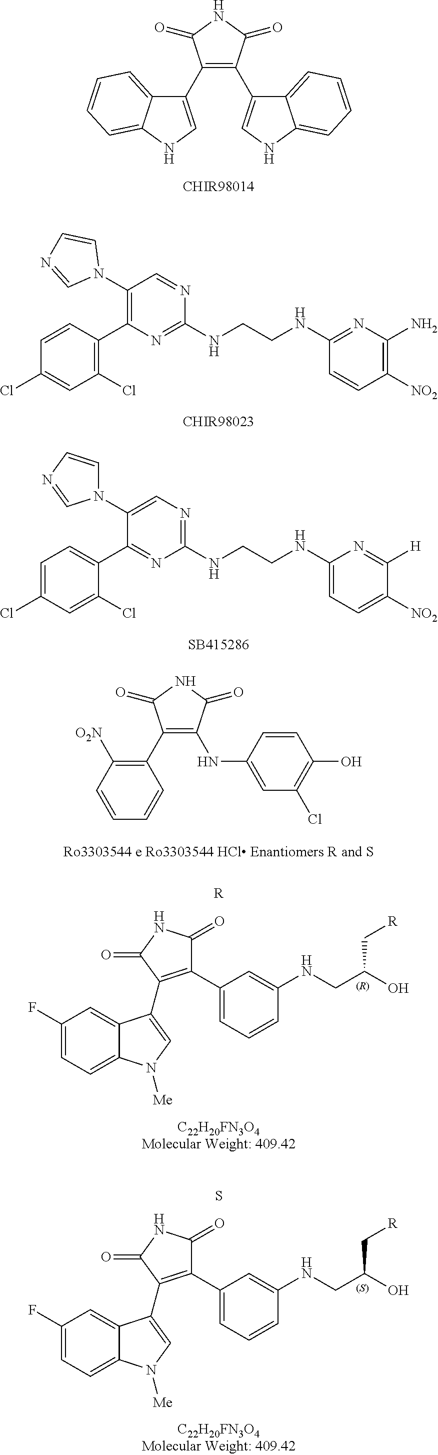

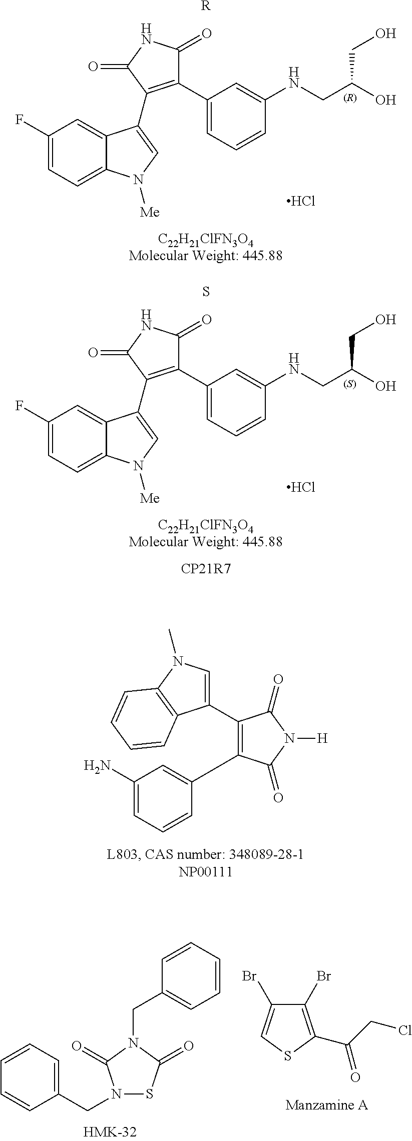

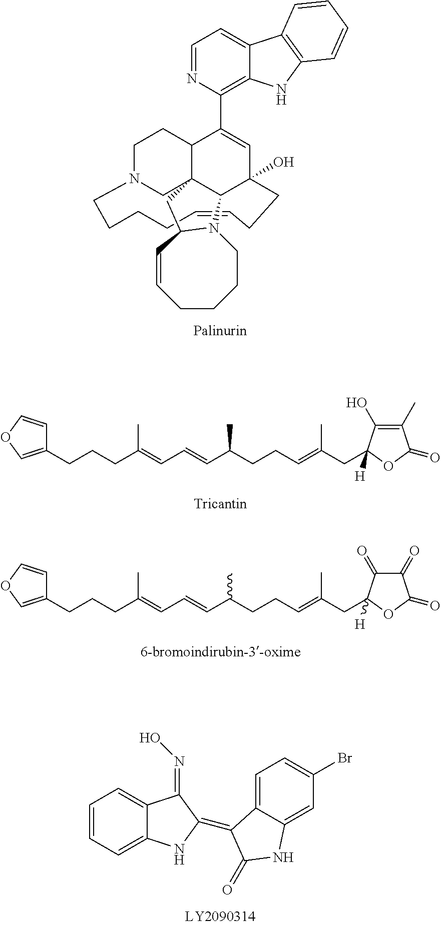 [SMILES: O[C@]12C=C(c3nccc4c3[nH]c3ccccc34)C3CCN(CCCC/C=C\CC1)C[C@@]31C[C@@H]3/C=C\CCCCN3[C@H]12 ; CC1=C(O)[C@@H](C/C(C)=C/CC[C@H](C)/C=C/C=C(\C)CCCc2ccoc2)OC1=O ; C/C(=C\C=C\C(C)CC/C=C(\C)CC1OC(=O)C(=O)C1=O)CCCc1ccoc1 ; O=C1Nc2cc(Br)ccc2/C1=C1/Nc2ccccc2/C1=N\O]

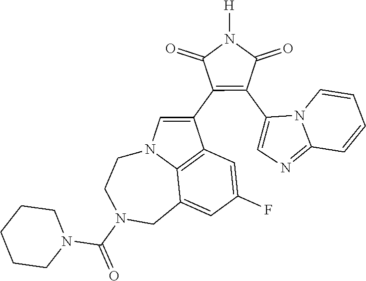 [SMILES: O=C1NC(=O)C(c2cnc3ccccn23)=C1c1cn2c3c(cc(F)cc13)CN(C(=O)N1CCCCC1)CC2]

One or more kinds of GSK-3 inhibitors may be used individually or in combination.

The GSK-3 inhibitors may be simultaneously added to a medium, or may be separately added to a medium in a staggered manner as long as the cultivation of the cells proceeds.

The medium which is added with the GSK-3 inhibitors is not particularly limited as long as it contains amino acids, physiologic salt concentrations and water, and is generally a medium used for cultivating cells (hereinafter to be also referred to as a basal medium for convenience). The basal medium is not particularly limited as long as it can be used for culturing animal cells, and includes, for example and not exclusively, MEM medium, BME medium, BGJb medium, CMRL 1066 medium, Glasgow MEM medium, Improved MEM Zinc Option medium, IMDM medium, Medium 199 medium, Eagle MEM medium, αMEM medium, DMEM medium, ham medium, RPMI 1640 medium, Fischer's medium, and a mixed medium thereof. These media are commercially available. Furthermore, the medium to be used in this step can be a serum-containing medium or serum-free medium, preferably a serum-free medium. When the medium to be used in this step is a serum-containing medium, mammalian sera such as bovine serum, fetal bovine serum, human serum and the like can be used. The concentration of the serum in a medium is 0.1-20%, preferably 1-10%.

The basal medium to be used in this step is preferably MEM medium.

Where necessary, the medium is exchanged as appropriate (e.g., once every 3 days). As mentioned above, the kind (combination) of the inhibitors to be added can be or is preferably changed as necessary.

Reversing and/or reducing and/or inhibiting of the endothelial to mesenchymal transition (EnMT) can be confirmed by measuring the expression and localization of markers suggestive of EnMT such as e.g. ZO-1 and/or a α-SMA and the like, in addition to the change of cell morphology.

In particular, the endothelial to mesenchymal transition (EnMT) is reversed and/or reduces and/or inhibited the expression and/or localization of ZO-1 is upregulated while the expression and/or localization of α-SMA is downregulated.

ZO-1 is preferably characterized by the sequences discloses in NCBI with the following Accession nos.: Protein Accession NCBI: NP_001287954.2, Uniprot: Q07157.3;

α-SMA is preferably characterized by the sequences discloses in NCBI with the following Accession nos.: Protein Accession NCBI: NP_001091.1, Uniprot: P68133.1.

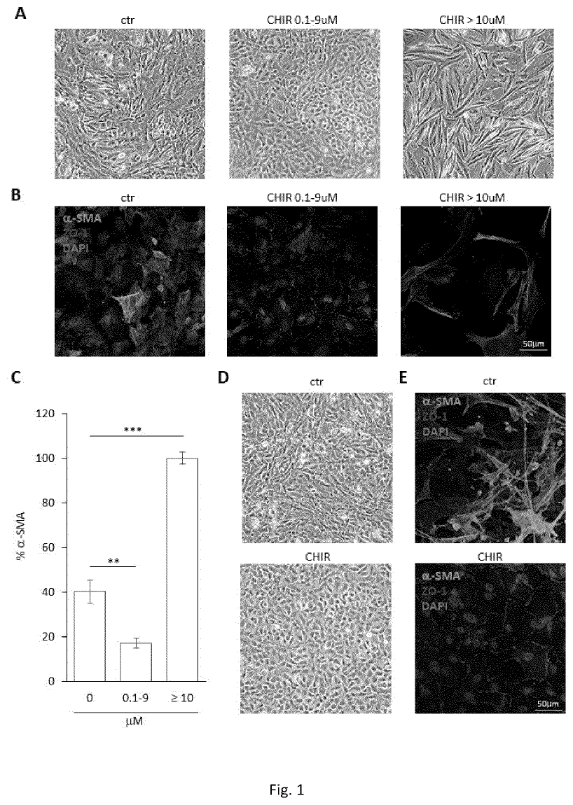

It can be evaluated the presence or absence of the expression of a protein/cell morphology necessary for exerting the function of corneal endothelial cells or a gene encoding same (corneal endothelial cell marker) (see for example FIG. 1). The expression of the marker or protein can be evaluated by a method utilizing an antigen antibody reaction and the like, and the expression of the marker or gene can be evaluated by a method utilizing RT-PCR and the like. The cell morphology can be evaluated under any microscope as a comparison with the reference figure. Conveniently, reversing and/or reducing and/or inhibiting of the endothelial to mesenchymal transition (EnMT) can also be confirmed by evaluating the cell morphology. In particular, a cell wherein the endothelial to mesenchymal transition (EnMT) is reversed and/or reduced and/or inhibited is characterized by a polygonal morphology upon reaching confluence, loosing the spindle-shaped morphology, associated with fibroblastic cells that had undergone EnMT (Català et al., 2021). Moreover, a cell wherein the EnMT is reversed and/or reduced and/or inhibited presents a clear expression and localization of typical markers such as ZO-1, while EnMT markers such as α-SMA result with a reduced expression and delocalized.

The method of the present invention allows to obtain corneal endothelial cells in large amounts. The obtained corneal endothelial cells have the correct cell shape to form a sealed continuous corneal endothelium to sustain the physiological function. This finding is confirmed by their correct morphology, behavior and protein expression and localization (for example, but not only, ZO-1, aSMA, Na+/K+ ATPase and others).

The obtained corneal endothelial cells can be utilized as a medicament such as a corneal endothelial cell sheet or cell suspension for corneal transplantation and the like.

The present invention provides a medicament containing corneal endothelial cells produced by the above-mentioned method of the present invention.

A corneal endothelial cell sheet obtained by the present invention can be used as a graft and/or cell suspension for the treatment of a disease requiring transplantation of corneal endothelium, for example, bullous keratopathy, corneal edema, corneal leukoma and the like.

In another embodiment other than a sheet, the obtained corneal endothelial cells as they are, a cell mass such as cell suspension obtained by concentration and the like, and the like are used as the medicament of the present invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a protein” includes a plurality of such proteins, and so forth.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the invention are specifically embraced by the present invention, as they are interconnected, and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The invention will be now illustrated by means of non-limiting examples referring to the following figures.

FIG. 1. CHIR99021 (CHIR) titration in primary cultures of human corneal endothelial cells (HCEnCs). A) Phase-contrast images of primary HCEnCs culture at passage1 (P1) treated with CHIR at different concentrations (0.1-10 μM), showing the effects of CHIR on their morphology if compared with the untreated HCEnCs (ctr). B) Immunofluorescence microscopy images of HCEnCs at P1 treated with CHIR at different concentrations (0.1-10 μM), showing expression of ZO-1 and α-SMA markers. DAPI counterstains nuclei. Scale bar 50 μm. C) Quantification of the percentage of HCEnCs treated with CHIR at different concentrations (0.1-10 μM) expressing α-SMA protein as seen in FIG. 1B. D) Phase-contrast images of primary HCEnCs culture at P3 treated with CHIR at 0.1-9 μM, showing the effects of CHIR on their morphology if compared with the untreated HCEnCs (ctr). E) Immunofluorescence microscopy images of HCEnCs at P3 treated with CHIR at 0.1-9 μM, showing expression of ZO-1 and α-SMA markers. DAPI counterstains nuclei. Scale bar 50 μm.

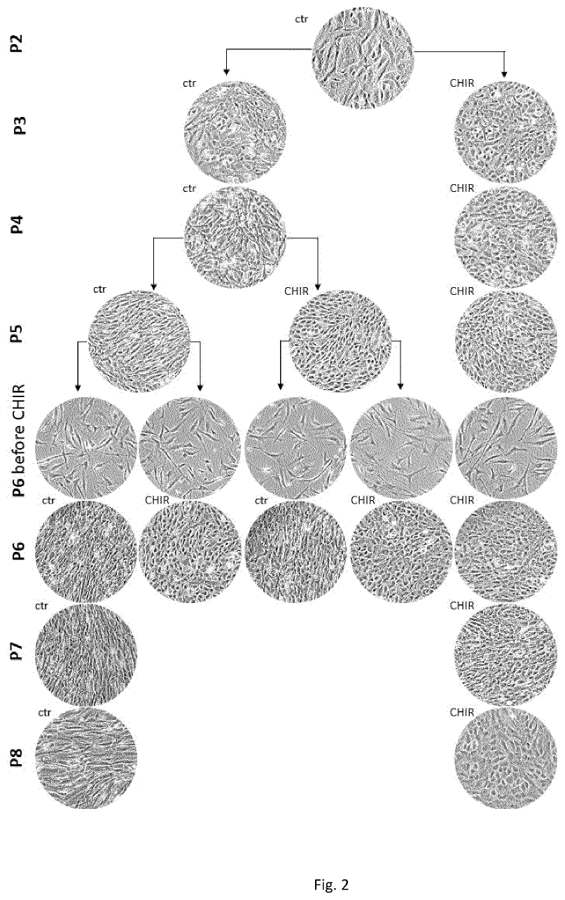

FIG. 2. CHIR reverses the mesenchymal phenotype in primary cultures of HCEnCs. Phase-contrast images of primary HCEnCs culture at progressive passages (P2 to P8) treated with CHIR at 0.1-9 μM, showing the effects of CHIR on their morphology if compared with the untreated HCEnCs (ctr). CHIR was added to the HCEnCs cultures after their morphology was already elongated at P2.

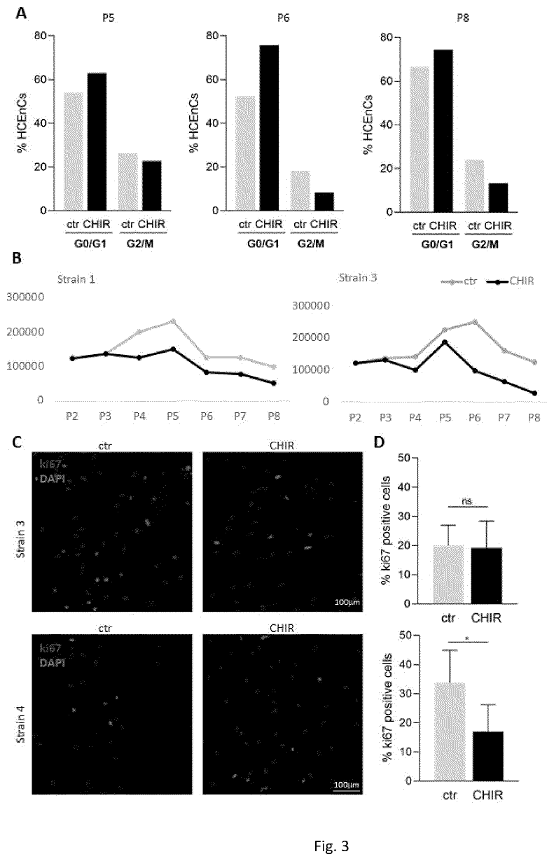

FIG. 3. CHIR reduces cell proliferation rate in primary cultures of HCEnCs. A) Fluorescence Activated Cells Sorting (FACS) analysis of primary HCEnCs at P5-P6-P8 treated with CHIR and compared with untreated HCEnCs (ctr), showing the effects of CHIR at 0.1-9 μM on cell proliferation. B) HCEnCs count done upon detachment at confluency for progressive passages, compared between HCEnCs treated with CHIR at 0.1-9 μM and untreated HCEnCs (ctr). C) Immunofluorescence microscopy images of sub-confluent HCEnCs at P5 untreated (ctr) or treated with CHIR at 0.1-9 M, showing expression of ki67 marker. DAPI counterstains nuclei. Scale bar 100 μm. D) Quantification of the HCEnCs expressing ki67 protein percentage at P5 untreated (ctr) or treated with CHIR at 0.1-9 μM as seen in FIG. 3C.

### EXAMPLES

### Materials and Methods

### Primary HCEnCs Culture

Human corneas tissues (obtained after informed consent to the eye bank and the use was approved by the ethical committee) were preserved in media for tissue preservation as for example Eusol, OptiSOL or others and used within approximately 15 days from death; a list of detail for some corneas used and relative experiments is shown in Table 1. HCEnCs were isolated through Descemet's stripping and digestion with Collagenase A (Roche) for 1-16 h at 30-39° C. After 5-10 minutes in Trypsin or other proteolytic enzymes at 37° C., HCEnCs were pelleted at 1,200 rpm for 3 min and plated in FNC Coating mix treated wells.

HCEnCs were cultured between 31 and 39° C. in 4-10% CO₂, changing the growth medium every other day. The growth medium can be OptiMEM-I, MEM, F12, Human Endothelial SFM, 2-10% HyClone fetal bovine serum (FBS), 2-10 ng/ml epidermal growth factor (EGF), 5-50 μg/mL ascorbic acid, 40-300 mg/L calcium chloride, 0.01-0.2% chondroitin sulphate, and penicillin/streptomycin. Upon confluency, HCEnCs were rinsed in DPBS and passaged at a ratio of 1:2 or 1:3 with Trypsin or other proteolytic enzymes for 10-30 min at 31-39° C. in 4-10% CO₂. Sub-confluent cultures were harvested approximately 1 day after plating.

TABLE 1
Some donor human corneas used for the experiments. List
of some donor human corneas details and the relative primary
HCEnCs strains used for experiments. Donor human corneas
features provided by the eye bank include Age (in years), D/P,
indicating the interval (in hours) between the death and the preservation
of these human corneas and the HCEnCs count (in cell/mm²),
done at the time of preservation. On the right, the columns
indicate the experiments carried out for these strains:
passages in culture (P2-P8), immunofluorescence analysis
(IF), FACS analysis.

| Primary | | | | | | |
|---|---|---|---|---|---|---|
| HCEnCs | Age | D/P | HCEnCs | Experiments | | |
| Strain | (y) | (h) | (cell/mm²) | Culture | IF | FACS |
| 1 | 68 | 20 | — | • P8 | • ZO1/α-SMA | • |
| 2 | 81 | 3 | 2215 | • P2 | • ZO1/α-SMA | |
| 3 | 51 | 18 | 3067-3226 | • P8 | • ZO1/α-SMA | • |
| | | | | | • ki67/α-SMA | |
| 4 | 26 | 21 | 3268 | • P6 | • ZO1/α-SMA | |
| | | | | | • ki67/α-SMA | |

### GSK Inhibitors Preparation and Addition to the Culture Media

Before or whenever primary HCEnCs encountered a morphological variation, the GSK inhibitor CHIR99021 (also defined as 6-[[2-[[4-(2,4-Dichlorophenyl)-5-(5-methyl-1H-imidazol-2-yl)-2-pyrimidinyl]amino]ethyl]amino]-3-pyridinecarbonitrile) was prepared and added to the culture media at a concentration range of 0.1-10 μM. Multiple solvents and methods have been used to dissolve CHIR stock solution. Ultrasonic were applied for CHIR dilutions in sodium carboxymethyl cellulose (CMC-Na) in saline water and 20% Sulfobutylether-β-Cyclodextrin (SBE-β-CD), adjusted to pH 3-6, with 1 N acetic acid. CHIR can be also diluted in dimethyl sulfoxide (DMSO) at 70-100% (v/v), with a solution composed of 10% DMSO 40% PEG300 5% Tween-80 in saline water or composed of 10% DMSO and 20% SBE-β-CD in saline water. Once obtained a clear solution, the stock solution was aliquoted and stored at-20/−80° C. until use.

### Immunofluorescence

Immunofluorescence staining was performed on primary cultured HCEnCs after fixation in 3% PFA 15 min at room temperature (RT). Triton X-100 used at 1% for 10 min at RT allowed cell permeabilization and a solution of bovine serum albumin (BSA) 2%, FBS 2%, Triton X-100 at 0.01% in PBS was used for 30 min at 37° C. to block the non-specific binding sites. Primary and secondary antibodies were incubated for 1 h at 37° C. while nuclei were counterstained with DAPI at RT for 5 minutes before mounting the glass coverslips using amounting medium such as DAKO. Primary antibodies used herein are listed in Table 2 while secondary antibodies used are: Alexa Fluor 488 anti-rabbit, 1:2,000, and Alexa Fluor 568 anti-mouse, 1:1,000 (Thermo Fisher Scientific). Images were obtained with a confocal microscope (LSM900 Airyscan—Carl Zeiss).

TABLE 2
Antibodies and relative dilutions used for IF analysis.

| Protein | Reference | dilution |
|---|---|---|
| ZO-1 | 40-2200 (Thermo Fisher) | 1:100 |
| α-SMA | A5228 (Sigma Aldrich) | 1:100 |
| Ki67 | Ab15580 (Abcam) | 1:50 |

### Cell Cycle Analysis by FACS

The cell cycle was studied by staining sub-confluent cultures of HCEnCs with Propidium Iodide (PI; Sigma-Aldrich). HCEnCs from cell culture were washed with DPBS and incubated for 1 h at 4° C. in the dark with 250 μL of a PBS solution composed by PI 50 μg/mL, Triton X-100 (Bio-Rad, USA) at 0.1%. Cells were then analysed using BD FACSCanto II (BD BIOSCIENCES; San Jose, CA USA). For each sample, 20,000 events were considered for the analysis to ensure statistical relevance and results were analysed with a ModFit 3.0 software.

### Results

### CHIR99021 (CHIR) Titration in Primary Cultures of Human Corneal Endothelial Cells (HCEnCs).

Primary HCEnCs culture at passage1 (P1) were treated with CHIR at different concentrations between 0.1 μM and 10 μM (FIG. 1). A reversion of HCEnCs elongated morphology was observed in cells treated with CHIR at 0.1-9 μM, which restored the polygonal morphology if compared to the untreated HCEnCs (ctr). A Mock control, where HCEnCs were treated with the solvent used to dilute CHIR, was done each time in parallel and showed no morphological differences with the untreated HCEnCs (ctr). HCEnCs treated with CHIR at concentrations >10 μM showed an exacerbated elongated morphology in culture when compared to untreated cells (FIG. 1A).

Protein expression of ZO-1 and α-SMA markers in HCEnCs at P1 was evaluated through immunocytochemistry analysis: cells treated with CHIR at 0.1-9 μM showed a restored sealed ZO-1 pattern of expression and concomitant reduced levels of α-SMA, if compared to the untreated HCEnCs (ctr). CHIR used at a concentration >10 μM accelerated the process of endothelial to mesenchymal transition (EnMT), as observed from α-SMA, which was highly expressed in every cell, and from the parallel disappearance of ZO-1 expression (FIG. 1B).

Quantification of the percentage of HCEnCs expressing α-SMA in immunofluorescence shown in FIG. 1B demonstrates that CHIR tested at the above-mentioned range of 0.1-9 μM was responsible for a significant reduction in the percentage of α-SMA expressing cells from 40% to 17% approximately (FIG. 1C). CHIR used at 0.1-9 μM was chosen therefore for all the subsequent experiments and will be indicated from now onwards as CHIR.

CHIR effects on cell morphology was further observed at P3: untreated cells (ctr) appeared more elongated than CHIR treated HCEnCs (FIG. 1D). HCEnCs at the same P3 were analysed for ZO-1 and α-SMA markers expression, obtaining similar results to those observed at P1: CHIR treatment drastically reduces expression of α-SMA and concomitantly restores ZO-1 pattern of tight junctions in the HCEnCs population (FIG. 1E).

CHIR demonstrated here the capacity to reduce the process of EnMT in primary HCEnCs, which hampers the opportunity to be amplified in culture and thus to be used for a regenerative medicine cell therapy approach.

### CHIR Reverses the Mesenchymal Phenotype in Primary Cultures of HCEnCs.

CHIR treatment was applied to primary HCEnCs already presenting an elongated morphology in culture (P3) and demonstrated capable of reverting the cell phenotype and restoring a polygonal morphology that characterize the corneal endothelium in vivo (FIG. 2).

Primary HCEnCs were cultured until P8. At P6, images of untreated (ctr) and CHIR treated HCEnCs has been shown the day after plating, before adding CHIR to the culture media, showing how, at any passage, the cells appeared with a similar morphology. CHIR treatment of HCEnCs was able to reverse the elongated morphology even whenever added at a later passage, as observed at P6 confluence. The CHIR effect of reversing the elongated phenotype on primary HCEnCs in culture could be appreciated at least up to P8.

The results obtained here highlight that primary HCEnCs, once acquired an elongated morphology in culture, can restore their characteristic polygonal morphology if stimulated with CHIR for subsequent passages in culture.

### CHIR Reduces Cell Proliferation Rate in Primary Cultures of HCEnCs.

Multiple assays on primary HCEnCs at subsequent passages demonstrated that CHIR addition to the culture media does not increase their proliferation rate (FIG. 3).

Fluorescence Activated Cells Sorting (FACS) analysis was performed on sub-confluent HCEnCs at P5-P6-P8, when HCEnCs morphology of the untreated control was elongated (as observed in FIG. 2). FACS analysis revealed that CHIR addition to the culture media enhanced the percentage of cells in G0/G1 phase of cell cycle and concomitantly reduced the percentage of cells at G2/M at subsequent passages in culture when compared to the untreated cells (FIG. 3A).

A reduction in cell proliferation rate upon CHIR treatment was confirmed by cell counting at confluency for each passage: the number of CHIR treated HCEnCs was constantly lower if compared with their relative untreated control (FIG. 3B).

Immunofluorescence analysis on sub-confluent HCEnCs at P5 showed an equal or lower amount of ki67 expressing cells in CHIR treated than in untreated cells, the latter exhibiting an elongated morphology at P5 (FIG. 3C). These images were quantified and confirmed an equal (Strain 3) or significant reduction (Strain 4, p<0.05) in ki67 protein expressing cells (FIG. 3D). CHIR addition in primary HCEnCs cultures does not enhance cell proliferation rate as evaluated with different assays.

### DISCUSSION

Inhibition of GSK-3 through the addition of CHIR99021 or analogues in culture, alone or in combination (data not shown), allowed for the first time to reverse the elongated phenotype (phenotype described by Frausto et al., 2020) that primary HCEnCs encounter in vitro after the second or third passage. The GSK-3 inhibition effect on cell morphology has been observed in HCEnCs derived now from multiple donors and for several subsequent passages in culture (FIG. 2). A titration of such compounds elicited a precise evaluation of their effect on HCEnCs morphology and expression markers and a consequent selection of the most appropriate concentrations range for obtaining a proper reversion of the mesenchymal phenotype (FIG. 1). Human primary HCEnCs have never been previously reversed from the elongated phenotype and GSK-3 inhibitors have never been previously used to reverse the mesenchymal phenotype, process that still hampers those cells to be maintained in culture for physiological applications. Pathway regulation of HCEnCs upon GSK-3 inhibitors treatment was previously studied by different research groups, including our group, when CHIR was applied to rabbit CEnCs (Maurizi et al., 2020), even though we did not describe this specific effect on cell morphology and EnMT. Moreover, CHIR was proven to prevent mesenchymal conversion (but not to reverse it) only in a corneal endothelial derived cell line, B4G12 (Wang et al., 2022). However, the B4G12 cell line is not comparable to a corneal endothelial primary culture or tissue as presenting a completely different expression pattern, in particular for cell cycle genes and protein expression (Thi et al., 2014). Moreover, the same group showed a concomitant enhancement in cell proliferation in B4G12, which we have never observed in CHIR treated primary HCEnCs with different assays (FIG. 3).

The GSK-3 inhibitors effect on multiple passages of primary HCEnCs would bring a substantial advantage in the capacity to maintain these cells in culture until late passages, preserving the correct morphology and markers expression, while overcoming EnMT. Maintenance of the correct polygonal phenotype and marker expression by using a single GSK-3 inhibitor is an important achievement for obtaining a quality product that will be used for the in vitro study of specific therapies and in a future clinical application. Further investigation is required for a deeper analysis on molecular pathways activation and marker expression upon GSK-3 inhibitors treatment.

### REFERENCES

Arras, W., H. Vercammen, S. Ni Dhubhghaill, C. Koppen, and B. Van den Bogerd. 2021. Proliferation increasing genetic engineering in human corneal endothelial cells: a literature review. Frontiers in Medicine. 8:688223.
Català, P., G. Thuret, H. Skottman, J. S. Mehta, M. Parekh, S. N. Dhubhghaill, R. W. Collin, R. M. Nuijts, S. Ferrari, and V. L. LaPointe. 2021. Approaches for corneal endothelium regenerative medicine. Progress in retinal and eye research: 100987.
Eveleth, D., S. Pizzuto, J. Weant, J. Jenkins-Eveleth, and R. A. Bradshaw. 2020. Proliferation of human corneal endothelia in organ culture stimulated by wounding and the engineered human fibroblast growth factor 1 derivative TTHX1114. Journal of Ocular Pharmacology and Therapeutics. 36:686-696.
Eveleth, D., and T. Tremblay. 2022. A Phase 1/Phase 2 Study Evaluating the Safety and Efficacy of TTHX1114 on the Regeneration of Corneal Endothelial Cells in Patients with Corneal Endothelial Dystrophy. Investigative Ophthalmology & Visual Science. 63:2757-A0246-2757-A0246.
Frausto, R. F., V. S. Swamy, G. S. Peh, P. M. Boere, E. M. Hanser, D. Chung, B. L. George, M. Morselli, L. Kao, and R. Azimov. 2020. Phenotypic and functional characterization of corneal endothelial cells during in vitro expansion. Scientific reports. 10:1-22.
Gain, P., R. Jullienne, Z. He, M. Aldossary, S. Acquart, F. Cognasse, and G. Thuret. 2016. Global Survey of Corneal Transplantation and Eye Banking. JAMA ophthalmology. 134:167-173.
Maurizi, E., D. Schiroli, R. Zini, A. Limongelli, R. Mistò, C. Macaluso, and G. Pellegrini. 2020. A fine-tuned β-catenin regulation during proliferation of corneal endothelial cells revealed using proteomics analysis. Scientific reports. 10:1-16.
Pizzuto, S., G. Duffey, J. Weant, and D. Eveleth. 2022a. Acceleration of Regeneration of the Corneal Endothelial Layer After Descemet Stripping Induced by the Engineered FGF TTHX1114 in Human Corneas in Organ Culture. Cornea: 10.1097.
Pizzuto, S., G. Duffey, J. Weant, and D. Eveleth. 2022b. A Human Corneal Organ Culture Model of Descemet's Stripping Only with Accelerated Healing Stimulated by Engineered Fibroblast Growth Factor 1. J. Vis. Exp. 185: e63482.
Thi, B. M. H., N. Campolmi, Z. He, A. Pipparelli, C. Manissolle, J.-Y. Thuret, S. Piselli, F. Forest, M. Peoc′h, and O. Garraud. 2014. Microarray analysis of cell cycle gene expression in adult human corneal endothelial cells. PLOS One. 9.
Wang, Y., C. Jin, H. Tian, J. Xu, J. Chen, S. Hu, Q. Li, L. Lu, Q. Ou, and G.-t. Xu. 2022. CHIR99021 balance TGFβ1 induced human corneal endothelial-to-mesenchymal transition to favor corneal endothelial cell proliferation. Experimental eye research. 219:108939.
Weant, J., D. D. Eveleth, A. Subramaniam, J. Jenkins-Eveleth, M. Blaber, L. Li, D. M. Ornitz, A. Alimardanov, T. Broadt, and H. Dong. 2021. Regenerative responses of rabbit corneal endothelial cells to stimulation by fibroblast growth factor 1 (FGF1) derivatives, TTHX1001 and TTHX1114. Growth Factors: 1-14.

1. An in vitro or ex vivo method for reversing and/or reducing and/or inhibiting the endothelial to mesenchymal transition (EnMT) process in corneal endothelial cells, said method comprising incubating said cells with an inhibitor of glycogen synthase kinase 3 (GSK-3).

2. The method according to claim 1 wherein said corneal endothelial cells are primary and/or human corneal endothelial cells.

3. The method according to claim 1 wherein said corneal endothelial cells are primary corneal endothelial cells.

4. The method according to claim 3 wherein said primary corneal endothelial cells are primary human corneal endothelial cells (HCEnCs).

5. The method according to claim 1 wherein the method does not further comprise the addition of any molecule able to induce endothelial to mesenchymal transition (EnMT).

6. An in vitro or ex vivo method for culturing and/or expanding and/or regenerating corneal endothelial cells comprising the method of claim 1.

7. A method of reversing and/or reducing and/or inhibiting the endothelial to mesenchymal transition (EnMT) process comprising contacting endothelial cells with an inhibitor of glycogen synthase kinase 3 (GSK-3).

8. A method for the treatment of a disease or condition associated to dysfunctional or damaged corneal endothelial cells, comprising contacting the dysfunctional or damaged endothelial cells with an inhibitor of glycogen synthase kinase 3 (GSK-3).

9. A method for reversing and/or reducing and/or inhibiting the EnMT process in corneal endothelial cells comprising contacting the corneal endothelial cells in need of such reducing and/or inhibiting with an inhibitor of glycogen synthase kinase 3 (GSK-3).

10. (canceled)

11. The method according to claim 1 wherein the inhibitor is selected from the group consisting of: CHIR99021, LiCl, Li2CO3, BIO ((2′Z,3′E)-6-bromoindirubin-3′-oxime), TD114-2, campalone, TWS119, CBM1078, SB216763, 3F8 (TOCRIS), AR-A014418, FRATide, indirubin 3′-oxime, CHIR98014, Kenpaullone, TDZD-8, BIO-acetoxime, Bis-7-indolylmaleimide, SB415286, Ro3303544, CP21R7, LY2090314, and L803 or an analogue thereof, such as 6-bromoindirubin-3′-oxime (BIO),
optionally said inhibitor of GSK-3 is used in combination with a molecule that stimulates corneal endothelial cells proliferation and regeneration.

12. The method according to claim 1 wherein the inhibitor is at concentrations of 0.1-9 μM.

13. The method according to claim 1 wherein the cells are incubated with an inhibitor of GSK-3 during cell amplification for 2 hours-10 days.

14. The method according to claim 13 wherein the cells are primary HCEnCs are in culture and the incubation of said primary HCEnCs culture with an inhibitor of GSK-3 consists in the addition of the inhibitor to the culture media or culturing the cells with a culture medium comprising the inhibitor.

15. The method according to claim 1 wherein reversing and/or reducing and/or inhibiting the EnMT process comprises reversing cell mesenchymal phenotype to endothelial phenotype and/or restoring the cell phenotype to a cell polygonal or hexagonal morphology and/or reverting cell elongated morphology and/or a variation in the expression of EnMT markers, e.g. ZO-1 and/or α-SMA.

16. The method according to claim 1 wherein the cell mesenchymal phenotype is reversed to endothelial phenotype.

17. A medicament comprising the corneal endothelial cell obtainable by the method according to claim 1.

18. (canceled)

19. (canceled)

20. The method of claim 8, wherein the disease or condition is selected from the group consisting of: Fuch's dystrophy, iridocorneal endothelial syndrome, posterior polymorphic dystrophy, congenital hereditary endothelial corneal dystrophy, corneal dystrophy, and advanced endothelial failure in corneal transplantation.

21. The method of claim 11, wherein said molecule is EGF or EGF analogue or FGF-2 or FGF-2 analogue.
